(19) United States
(12) Patent Application Publication
(10) Pub. No.: US 2010/0179151 A1
(43) Pub. Date: Jul. 15, 2010

(54) TRANSDERMAL APPLICATION OF TRIAZINES FOR CONTROLLING INFECTIONS WITH COCCIDIA

(75) Inventors: Iris Heep, Koeln (DE); Hans-Christian Mundt, Erkrath (DE)

Correspondence Address: BAYER HEALTHCARE LLC, P.O.BOX 390, SHAWNEE MISSION, KS, 66201, US

(73) Assignee: BAYER ANIMAL HEALTH GMBH, Leverkusen (DE)

(21) Appl. No.: 12/377,156

(22) PCT Filed: Aug. 8, 2007
(86) PCT No.: PCT/EP07/06992

§ 371 Date: Feb. 25, 2009

(30) Foreign Application Priority Data
Aug. 16, 2006 (DE) 10 2006 038 292.7

(51) Int. Cl. A61K 31/53; A61P 33/02

(52) U.S. Cl. 514/242; 514/241

(57) ABSTRACT

The present invention relates to the transdermal application of triazines such as toltrazuril or ponazuril for controlling infections with coccidia in humans and animals.

The present invention relates to the transdermal application of triazines such as toltrazuril or ponazuril for controlling infections with coccidia in humans and animals.

Coccidioses are frequently occurring parasitic infectious diseases in animals. Thus, for example, protozoans from the genera Eimeria, Isospora, Neospora, Sarcosporidia and Toxoplasma cause coccidioses all over the world. Examples of economically important coccidioses are: infections of pigs with coccidia of the genus Isospora or of cattle with coccidia of the genus Eimeria. Injections with Isospora suis have only in recent years been recognised as the cause of diarrhoea in piglets and have been studied intensively. As a rule, an infection proceeds from the environment to the piglets, or from piglet to piglet, via oocysts, which contain in each case two sporocysts with in each case two sporozoites. The parasitic stages multiply in the epithelial cells of the small intestine's villi, the presence of abenteric stages is the liver, spleen and lymph nodes is being discussed. The clinical picture of the disease includes a necrotic, inflammatory destruction of the gut's epithelial cells with atrophying villi, and, as a result, impaired absorption and digestion. The characteristic of an acute disease is an aqueous, whitish to yellow diarrhoea, which mostly occurs in week 2 to 3 of life. The weight gain of infected piglets is reduced. Treatment and therapy of the disease are insufficient to date. Antibiotics are ineffective; while sulphonamides are recommended, therapy is, as a rule, too late. Further treatment options are contradictory: the administration of, for example, monensin, amprolium or furazolidone has not been successful in preventing the disease in experimentally infected piglets. In more recent studies, Isospora suis has been identified in up to 92% of all litters in some farms, despite good hygiene.

Triazines, in particular toltrazuril and ponzazuril, and their activity against coccidia are known from a series of publications, see, inter alia, DE-A 27 18 799 and DE-A 24 137 22. WO 99/62519 discloses semi-solid aqueous preparations of toltrazuril-sulphone (ponazuril). It is also known that in particular toltrazuril is suitable for treating coccidiosis (for example Isospora suis) in pigs. See, for example, also the following publications: Don't forget coccidiosis, update on Isosporosis in piglets. Part I, Pig Progress volume 17, No2, 12-14; Mundt., H.-C., A. Daugschies, V. Letkova (2001): be aware of piglet coccidiosis diagnostics. Part II, Pig Progress volume 17, No 4, 18-20; Mundt, H.-C., G.-Pl Martineau, K. Larsen (2001): control of coccidiosis Part III, Pig Progress volume 17, No 6, 18-19.

Coccidioses in cattle by infection with various pathogenic Eimeria spp. (for example E. bovis and E. Zürnii) manifest themselves as cases of diarrhoea with different degrees of severity (bloody diarrhoeas accompanied by mortality).

In animals, the transdermal application of pharmaceuticals is particularly simple and convenient. In comparison with traditional, oral application, it is also advantageous in animals because transdermal application involves less stress for the animals. Since, however, it is frequently difficult to develop satisfactory transdermal formulations, the transdermal use in the control of infections with coccidia is as yet not conventionally used in practice. Accordingly, no commercial products of this type exist.

WO 96/38140, DE 10049468 or WO 00/37063 describe compositions against coccidiosis in animals. These publications generally also describe the external use, in addition to other types of use.

However, it is also known that the skin of animals differs markedly between the species and is therefore not always comparable in different animal species. Thus, it cannot be assumed that an active substance is transdermally effective in any animal species or even in humans if effectiveness has been found in just one animal species.

It has now been found that triazine active substances have systemic activity against infections with coccidia especially in animals, in particular mammals and here in particular productive mammals (agricultural livestock), even when the active substances are formulated as a formulation for transdermal application.

It is decisive for a transdermal formulation with efficacy in practice that a sufficiently high blood level of the active substance is achieved in the serum and that the active substances reach the pathogens. Full activity, combined with usual dosage rates, is desirable in this context.

Surprisingly, we have found that in the case of the triazines, full activity can be achieved with transdermal application even when only the dosage rate conventionally used for oral administration is employed. Usually, transdermal application requires a markedly higher dose than for example oral application. As a rule, the skilled worker expects at least a multiple of the oral dose for the dose required for dermal application (J. H. Vaile and P. Davis, Topical NSAIDs for musculoskeletal conditions, Drugs, 1998 Nov., 56 (5), 783-799. G. Graziani, G. A. Abbiati, E. Dolfini, R. Testa and G. P. Velo, Pharmacokinetic, Pharmacodynamic, and toxicological properties of naproxen gel in laboratory animals, Current therapeutic research, Sept. 1987, 42 (3), 480-490. 3 P. Clays, A. Barel, J. Taeymans, Perkutane Penetration von Medikamenten-Anwendung in der Physiotherapie, Sportverletzungen and Sportschaden [Percutaneous penetration of medicament administration in physiotherapy, sports injuries and sports damage], Sportphysiotherapie aktuell, Dec. 1997, 19-23).

Thus, it has been demonstrated that transdermal formulations of the active substances have made it possible to achieve serum levels required for an activity against infections with coccidia in rabbits, pigs and cattle.

For example, the pour-on administration of formulations with toltrazuril has resulted in the percutaneous uptake by all animals of the active substance. Astonishingly, this is even the case in animal species with little hair. Animals with little hair, such as pigs, ensure the protective function of the outer integument via a thicker epidermis. It is entirely surprising that the active substance is absorbed across this thicker skin and in particular also across the stratum corneum of the skin, which is particularly thick in pigs. Moreover, the active substance can also not be taken up via the large number of hair follicles, as is in the case in other animal species with a thicker coat of hair. Thus, it was surprising that, in studies on pigs, a massive infection with Isospora suis, the causative agent of coccidiosis in suckling pigs, was successfully controlled by means of a pour-on treatment with toltrazuril. The efficacy was demonstrated both clinically (reduced incidence of diarrhoea, better weight development) and parasitologically (reduced elimination of oocysts). Besides, the percutaneous absorption was also demonstrated by detecting the active substance and its main metabolite in various body tissues (serum, musculature, skin, liver, kidney).

The invention therefore relates to the use of triazines of the formula (I) or (II)

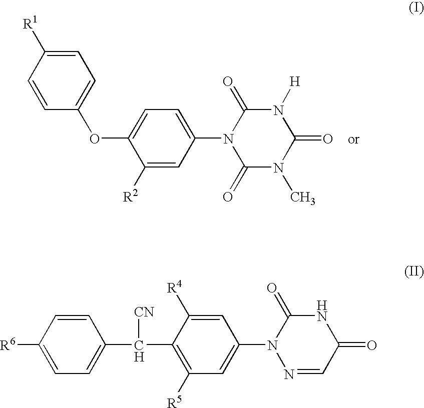

where

R¹ represents R³—SO₂— or R³—S—,

R² represents alkyl, alkoxy, halogen or SO₂N(CH₃)₂ and

R³ represents haloalkyl

R⁴ and R⁵ independently of one another represent hydrogen or Cl and

R⁶ represents fluorine or chlorine

and their physiologically acceptable salts

for the preparation of compositions for the transdermal treatment of infections of animals or humans with coccidia.

The triazines are well known per se as active substances against infections with coccidia; substances which may be mentioned are the triazinetriones such as, for example, toltrazuril and ponazuril, and the triazinediones such as, for example, clazuril, diclazuril and letrazuril.

The triazinediones are represented by the formula (II):

clazuril (R⁴═Cl, R⁵═H, R⁶═Cl in formula (II))

letrazuril (R⁴═Cl, R⁵═Cl, R⁶═F in formula (II)) and

diclazuril (R⁴═Cl, R⁵═Cl, R⁶═Cl in formula (II)).

Among these 1,2,4-triazinediones, diclazuril is most preferred.

Especially preferred according to the invention are the triazinetriones of the formula (I) as active substances:
R² preferably represents alkyl or alkoxy, each of which has up to 4 carbon atoms, especially preferably methyl, ethyl, n-propyl, i-propyl.
R³ preferably represents perfluoroalkyl having 1 to 3 carbon atoms, especially preferably trifluoromethyl or pentafluoroethyl.

The preferred triazinetriones are represented by formula (I):

toltrazuril (R¹═R³—S—, R²═CH₃, R³═CF₃)

ponazuril (R¹═R³—SO₂—, R²═CH₃, R³═CF₃)

Combinations with other active substances are also possible, for example with those which are employed for other indications, such as, for example, anthelmintics, antibiotics or dermal antiparasitics.

The dosage rate of the triazine—as has been mentioned above—may vary depending on the animal species. Conventional dosage rates are from 1 to 60 mg of active substance per kg bodyweight (mg/kg) of the animal to be treated per day, preferably 5 to 40 mg/kg and especially preferably 10 to 30 mg/kg.

In the case of the transdermal treatment according to the invention, the dosage rate may be approximately the same or lower than in the case of oral administration. In this context, “approximately the same or lower” is understood as meaning that the dermal dosage rate per day amounts to no more than 200%, preferably no more than 150%, especially preferably no more than 110%, in particular no more than 100%, of the corresponding oral dosage rate under otherwise identical conditions.

For oral administration, toltrazuril is usually given at the following dosage rates:

pigs: 20 mg/kg bodyweight

cattle: 15 mg/kg bodyweight

sheep: 20 mg/kg bodyweight

15 mg/kg bodyweight

Except in poultry, toltrazuril is only administered once per treatment, so that for example in the case of pigs, cattle and sheep the dosage rates stated are both per day and per treatment. In poultry, the dose stated is divided between two successive days.

Preparations which are suitable for animals are: solutions, suspensions or emulsions which are applied as what are known as spot-on or pour-on formulations for example to the back or the neck of the animals. Solutions are preferred.

Pour-on or spot-on formulations are prepared by dissolving, suspending or emulsifying the active ingredient in suitable dermatologically acceptable solvents or solvent mixtures. If appropriate, further adjuvants such as solubilizers, absorption accelerators, antioxidants, preservatives, thickeners, adhesives, pH regulators, UV stabilizers or colorants are added.

Solvents which may be mentioned are: physiologically acceptable solvents such as water, alcohols, such as, for example, monohydric alkanols (for example ethanol or n-butanol), polyhydric alcohols such as glycols (for example ethylene glycol, propylene glycol), polyethylene glycols (for example tetraglycol), polypropylene glycols, glycerol; aromatically substituted alcohols such as benzyl alcohol, phenylethanol, phenoxyethanol; esters such as ethyl acetate, butyl acetate, benzyl benzoate, ethyl oleate; ethers such as alkylene glycol alkyl ethers (for example dipropylene glycol monomethyl ether, diethylene glycol monobutyl ether); ketones such as acetone, methyl ethyl ketone; aromatic and/or aliphatic hydrocarbons, vegetable or synthetic oils; glycerol formal, solketal (2,2-dimethyl-4-hydroxymethy1-1,3-dioxolane), N-methylpyrrolidone, 2-pyrrolidone, N,N-dimethylacetamide, glycofurol, dimethylisosorbitol, lauroglycol, propylene carbonate, octyldodecanol, dimethylformamide, and mixtures of the abovementioned solvents.

Solubilizers which may be mentioned are: solvents which promote the dissolution of the active ingredient in the main solvent or which prevent its precipitation. Examples are polyvinylpyrrolidone, polyoxyethylated castor oil, polyoxyethylated sorbitan esters.

Absorption accelerators are:
ionic substances such as, for example, sodium lauryl sulphate.
dialkyl sulphoxides such as, for example, dimethyl sulphoxide and decyl methyl sulphoxide.
omega-amino acids and their derivatives such as, for example, dodecylazacycloheptan-2-one (Azone®), N-dodecyl-2-pyrrolidone or dodecyloxycarbonylpentylammonium dodecyloxy-carbonylpentylcarbamate (Transkarbam 12).
dipolar aprotic solvents such as, for example, dimethylacetamide, dimethylformamide, 2-pyrrolidone, N-methylpyrrolidone.
aliphatic alcohols having 1 to 4 carbon atoms, such as ethanol or isopropanol.
polyalcohols such as glycerol or polyethylene glycol, propylene glycol, diethylene glycol or dipropylene glycol.
fatty alcohols such as, for example, dodecanol, oleyl alcohol or isostearyl alcohol.
esters and amides of organic carboxylic acids: for example short-chain esters such as ethyl acetate; fatty acid esters such as glycerol monolaurate, glycerol monooleate, oleyl oleate, propylene glycol diesters of caprylic/capric acid; esters comprising amino groups, such as dodecyl N,N-dimethylaminoacetate, or the capsaicin analogue nonivamide.
emulsifiers from the classes polyoxyethylene fatty alcohol ethers, for example polyoxyethylene glycerol monostearate, polysorbates, for example polyoxyethylene −20 sorbitan monooleate, or sorbitan fatty acid esters, for example sorbitan monolaurate
amines such as, for example, dodecylamine
urea or urea compounds
cyclic acetals, for example 2-nonyl-4-hydroxymethyldioxolane, 2-nonyl-1,3-dioxolane.
fatty acids such as oleic acid or lauric acid.
essential oils, in particular from the class of the terpenes, such as linolene, limonene, 1,8-cineol, nerolidol (C15) or menthol.
Spreading oils such as silicone oil, isopropyl myristate or isopropyl palmitate.
Triglycerides such as medium-chain triglycerides of chain length C₈-C₁₂

Antioxidants are sulphites or metabisulphites such as potassium metabisulphite or sodium metabisulphite, sodium disulphite or potassium disulphite, ascorbic acid, isoascorbic acid, ascorbyl palmitate, gallic acid esters, butylhydroxytoluene, butylhydroxyanisole or tocopherol.

Synergists of these antioxidants may be: amino acids (for example alanine, arginine, methionine, cysteine), citric acid, tartaric acid, edetic acid or their salts, phosphoric acid derivatives or polyalcohols (polyethylene glycol).

Preservatives are: benzyl alcohol, benzalkonium chloride, trichlorobutanol, p-hydroxybenzoate, n-butanol, chlorocresol, cresol, phenol, benzoic acid, citric acid, tartaric acid or sorbic acid.

Thickeners are: inorganic thickeners such as bentonites, silica (for example amorphous, colloidal or highly disperse silica), aluminium stearates, organic thickeners such as cellulose derivatives, for example Hydroxypropylmethylcellulose 4000, polyvinyl alcohols and their copolymers, acrylates and methacrylates.

Adhesives are, for example, cellulose derivatives, starch derivatives, polyacrylates, natural polymers such as alginates, gelatin.

pH regulators are pharmaceutically customary acids or bases. The bases include alkali metal hydroxides or alkaline earth metal hydroxides (for example NaOH, KOH), basic salts such as, for example, ammonium chloride, basic amino acids such as, for example, arginine, choline, meglumine, ethanolamines or else buffers such as tris(hydroxymethyl)aminomethane, citric acid buffers or phosphate buffers. The acids include, for example, hydrochloric acid, acetic acid, tartaric acid, citric acid, lactic acid, succinic acid, adipic acid, octanoic or linolenic acid such as acidic amino acids such as, for example, aspartic acid.

UV stabilizers are, for example, substances from the class of the benzophenones, or novantisolic acid.

Colorants are all colorants which are approved for use on humans or animals and which may be dissolved or suspended.

Emulsions as a pour-on or spot-on formulation are either of the water-in-oil type or of the oil-in-water type.

They are prepared by dissolving the active substance in one phase and homogenising this phase with the aid of suitable emulsifiers and, if appropriate, further adjuvants such as colorants, absorption accelerators, preservatives, antioxidants, UV stabilizers, viscosity-increasing substances.

The following may be mentioned as the hydrophobic phase (oils): liquid paraffins, silicone oils, natural vegetable oils such as sesame seed oil, almond oil, castor oil, synthetic triglycerides such as caprylic/capric acid biglyceride, triglyceride mixtures with vegetable fatty acids of chain length C₈₋₁₂ or other specifically selected natural fatty acids, partial glyceride mixtures of saturated or unsaturated, optionally also hydroxyl-containing fatty acids, mono- and diglycerides of the C₈/C₁₀-fatty acids.

Fatty acid esters such as ethyl stearate, di-n-butyryl adipate, hexyl laurate, dipropylene glycol pelargonate, esters of a branched fatty acid of medium chain length with saturated fatty alcohols of chain length C₁₆-C₁₈, isopropyl myristate, isopropyl palmitate, caprylic/capric esters of saturated fatty alcohols of chain length C₁₂-C₁₈, isopropyl stearate, oleyl oleate, decyl oleate, ethyl oleate, ethyl lactate, waxy fatty acid esters such as artificial duck uropygial fat, dibutyl phthalate, diisopropyl adipate, ester mixtures related to the latter, and the like.

Fatty alcohols such as isotridecyl alcohol, 2-octyldodecanol, cetylstearyl alcohol, oleyl alcohol.

Fatty acids such as for example oleic acid and its mixtures.

The following may be mentioned as the hydrophilic phase:

water, alcohols such as, for example, propylene glycol, glycerol, sorbitol and their mixtures.

The following may be mentioned as emulsifiers: surfactants (comprises emulsifiers and wetters), such as
1. nonionic surfactants, for example polyethoxylated castor oil, polyethoxylated sorbitan monooleate, sorbitan monostearate, ethyl alcohol, glycerol monostearate, polyoxyethyl stearate, alkylphenol polyglycol ethers,
2. ampholytic surfactants, such as disodium-N-lauryl β-iminodipropionate or lecithin,
3. anionic surfactants such as sodium lauryl sulphate, fatty alcohol ether sulphates, mono/dialkyl polyglycol ether orthophosphoric ester monoethanolamine salt,
4. cationic surfactants such as cetyltrimethylammonium chloride.

The following are suitable as further adjuvants:

Viscosity-increasing and emulsion-stabilizing substances such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silica, or mixtures of the above.

Suspensions as pour-on/spot-on formulation may also be employed cutaneously. They are prepared by suspending the active substance in a liquid vehicle, if appropriate with addition of further adjuvants such as wetters, colorants, absorption accelerators, thickeners, adhesives, preservatives, antioxidants or UV stabilizers.

Liquid vehicles which may be mentioned are all homogeneous solvents and solvent mixtures.

The following may be mentioned as wetters (dispersants):

surfactants (comprises emulsifiers and wetters) such as
1. anionic surfactants such as sodium lauryl sulphate, fatty alcohol ether sulphates, mono/dialkyl polyglycol ether orthophosphoric ester monoethanolamine salt, lignosulphonates or dioctylsulphosuccinate,
2. cationic surfactants such as cetyltrimethylammonium chloride,
3. ampholytic surfactants, such as disodium-N-lauryl β-iminodipropionate or lecithin,
4. nonionic surfactants, for example polyethoxylated castor oil, polyethoxylated sorbitan monooleate, sorbitan monostearate, ethyl alcohol, glycerol monostearate, polyoxyethylene stearate, alkylphenol polyglycol ethers, Pluronic®.

Further adjuvants which may be mentioned are those detailed hereinabove.

The active substances may also be applied in the form of an aerosol. To this end, the active substance is finely distributed in a suitable formulation under pressure.

Those which may be mentioned as being preferred are solutions for transdermal administration, comprising compounds of the formula (I) or (II), which are characterized in that
a) the active substance is present in a concentration of from 0.1-30% by weight, in particular from 2-25% by weight and specifically from 5-20% by weight,
b) if appropriate, they contain substances for regulating the pH
c) if appropriate, they contain substances for influencing the transdermal absorption in a concentration of from 0.1-50% by weight, especially 1-20% by weight and specifically 1-10% by weight,
d) if appropriate, they contain preservatives for a sufficient preservation, either singly or in combination with what are known as synergists. The preservatives are usually present in concentrations of from 0.01-5% by weight and specifically 0.05-1% by weight.
e) if appropriate, they contain antioxidants in a concentration of from 0.1 to 1% by weight,
f) the pH of the solution is 3-10, in particular 4-9 and specifically 5-8.

Solvents which may be used for these preferred solutions are those solvents which have been mentioned further above; preferred examples of solvents which may be mentioned are N-methylpyrrolidone and dimethylacetamide.

Substances for influencing the transdermal absorption (known as penetration enhancers) also have already been mentioned further above, preferred examples being isopropanol, dodecylazacycloheptan-2-one (Azone®), limonene and 1,8-cineol.

Antioxidants which are preferably employed in the abovementioned formulations are BHA or BHT. For sufficient preservation, the preservatives may be employed singly or else in combination with what are known as synergists. Synergists such as citric acid, tartaric acid, ascorbic acid or the sodium salt of edetic acid are usually present in concentrations of from 0.01-1% by weight, specifically 0.05-0.15% by weight.

As has already been mentioned further above, the preferred solutions may contain a thickener for adjusting the suitable consistency, specifically usually preferably in concentrations of from 0.5 to 2% by weight. An example of a preferred thickener which may be mentioned is hydroxypropylmethylcellulose.

It is preferred to use hydrochloric acid (for example 1N HCl) or sodium hydroxide solution (for example 1N NaOH) for adjusting the pH.

Systemically active pour-on/spot-on formulations for use on the skin or in body cavities are poured on, spotted on, spread on, splashed on, rubbed in, sprayed on or applied by bathing, during which process the active substance penetrates the skin and acts systemically. Preferred in accordance with the invention is the use of as small as possible a volume in the form of a pour-on or spot-on application.

While having a surprisingly low toxicity for warm-blooded species, the active substances are suitable for the control according to the invention of coccidia which are found in animal keeping and animal breeding in livestock, breeding animals, zoo animals, laboratory animals, experimental animals and companion animals. Here, they are effective against all or individual stages of development of the pests and against resistant and normally-sensitive strains. Control of the parasitic protozoa is intended to reduce disease, deaths and reduction in performance (for example in the production of meat, milk, wool, hides, eggs and the like) so that more economic and easier animal keeping is possible through the use of the active substances.

The coccidia include:

Mastigophora (Flagellata) such as, for example, Trypanosomatidae, for example Trypanosoma brucei, T. gambiense, T. rhodesiense, T. congolense, T. cruzi, T. evansi, T. equinum, T. lewisi, T. percae, T. simiae, T. vivax, Leishmania brasiliensis, L. donovani, L. tropica, such as, for example, Trichomonadidae, for example Giardia lamblia, G. canis.

Sarcomastigophora (Rhizopoda) such as Entamoebidae, for example Entamoeba histolytica, Hartmanellidae, for example Acanthamoeba sp., Hartmanella sp.

Apicomplexa (Sporozoa) such as Eimeridae, for example Eimeria acervulina, E. adenoides, E. alabahmensis, E. anatis, E. anseris, E. arloingi, E. ashata, E. auburnensis, E. bovis, E. brunetti, E. canis, E. chinchillae, E. clupearum, E. columbae, E. contorta, E. crandalis, E. debliecki, E. dispersa, E. ellipsoidales, E. falciformis, E. faurei, E. flavescens, E. gallopavonis, E. hagani, E. intestinalis, E. iroquoina, E. irresidua, E. labbeana, E. leucarti, E. magna, E. maxima, E. media, E. meleagridis, E. meleagrimitis, E. mitis, E. necatrix, E ninakohlyakimovae, E. ovis, E. parva, E. pavonis, E. perforans, E. phasani, E. piriformis, E. praecox, E. residua, E. scabra, E. spec., E. stiedai, E. suis, E. tenella, E. truncata, E. truttae, E. zuernii, Globidium spec., Isospora belli, I. canis, I. felis, I. ohioensis, I. rivolta, I. spec., I. suis, Neospora caninum, N. hugesi, Cystisospora spec., Cryptosporidium spec. such as Toxoplasmadidae, for example Toxoplasma gondii, such as Sarcocystidae, for example Sarcocystis bovicanis, S. bovihominis, S. neurona, S. ovicanis, S. ovifelis, S. spec., S. suihominis such as Leucozoidae, for example Leucozytozoon simondi, such as Plasmodiidae, for example Plasmodium berghei, P. falciparum, P. malariae, P. ovale, P. vivax, P. spec., such as Piroplasmea, for example Babesia argentina, B. bovis, B. canis, B. spec., Theileria parva, Theileria spec., such as Adeleina, for example Hepatozoon canis, H. spec.

Furthermore Myxospora and Microspora, for example Glugea spec., Nosema spec.

Furthermore pneumocystis carinii, and Ciliophora (Ciliata) such as, for example, Balantidium coli, Ichthiophthirius spec., Trichodina spec., Epistylis spec.

Those protozoan genera and species which in pigs lead to subclinical or clinical infections must be very especially emphasized, in particular: Eimeria debliecki, E. suis, E. scabra, E. perminuta, E. spinosa, E. polita, E. porci, E. neodebliecki, Isospora suis, Cryptosporidium, Toxoplasma gondii, Sarcocystis miescheriana, S. suihominis, Babesia trautmanni, B. perroncitoi, Balantidium coli.

The livestock and breeding animals include mammals such as, for example, cattle, horses, sheep, pigs, goats, camels, water buffalos, donkeys, rabbits, fallow deer, reindeer, fur bearers such as, for example, mink, chinchilla, racoon, birds such as, for example, chickens, geese, turkeys, ducks, pigeons, ostriches, and bird species for keeping in domestic premises and in zoos. They furthermore include farmed fish and ornamental fish. Particular emphasis may be placed on pigs, cattle, sheep and dogs in all species, subspecies and breeds.

The laboratory animals and experimental animals include mice, rats, guinea pigs, golden hamsters, dogs and cats.

The companion animals include dogs and cats.

The examples which follow are intended to illustrate the invention, but without imposing any limitation:

### EXAMPLES

I. Formulation Examples

Formulation 1
5% ponazuril or toltrazuril
0.1% butylhydroxyanisole to 100% N-methylpyrrolidone

Formulation 2
4% toltrazuril
0.1% butylhydroxyanisole to 100% Solketal

Formulation 3
10% ponazuril
2% benzyl alcohol
5% highly disperse silica (for example Aerosil 200) to 100% dimethylacetamide

Formulation 4
10% toltrazuril
3% n-butanol
0.1% butylhydroxyanisole to 100% 2-pyrrolidone

Formulation 5
5% toltrazuril to 100% tetraglycol

Formulation 6
20% toltrazuril
0.8% hydroxypropylmethylcellulose 4000
79.2% N-methylpyrrolidone

Formulation 7
20% toltrazuril
0.8% hydroxypropylmethylcellulose 4000
79.2% dimethylacetamide

Formulation 8
20% toltrazuril
1% hydroxypropylmethylcellulose 4000
10% isopropanol
69% N-methylpyrrolidone

Formulation 9
20% toltrazuril
1% hydroxypropylmethylcellulose 4000
10% oleic acid
69% N-methylpyrrolidone

Formulation 10
5% toltrazuril to 100% N-methylpyrrolidone

Formulation 11
20% toltrazuril
1% hydroxypropylmethylcellulose 4000
10% dodecylazacycloheptan-2-one (Azone®) to 100% N-methylpyrrolidone

Formulation 12
20% toltrazuril
1% hydroxypropylmethylcellulose 4000
10% limonene to 100% N-methylpyrrolidone

Formulation 13
20% toltrazuril
1% hydroxypropylmethylcellulose 4000
10% 1,8-cineol to 100% N-methylpyrrolidone

The substances are mixed and stirred until a clear solution has formed. The antioxidants are first dissolved in the solvent, and the active substances are added in, and the thickeners are subsequently added. The preparation may also be effected in a different order, for example by first adding the thickener to the solvent, then adding and dissolving the antioxidant, if appropriate, and simultaneously or thereafter the active substance. Finally, the solutions may (do not have to) be filtered and are transferred into suitable containers.

II. Activity Studies in Animals:

A. Determination of the Serum Concentrations of Toltrazuril and Ponazuril after Pour-On Application in Calves and Rabbits

On day 0, the formulation of Example 10 was applied externally at a dose of 20 mg per kg (body weight) to in each case three calves and rabbits. At the points in time specified in the tables, the serum concentrations of toltrazuril and its metabolite ponazuril (toltrazuril sulphone) were determined. The results are shown in Tables 1 and 2.

TABLE 1
Serum values of toltrazuril/ponazuril in calves.

| | | Animal 1 | Animal 2 | Animal 3 |
|---|---|---|---|---|
| Toltrazuril in mg/l | 0 | <LoQ | <LoQ | <LoQ |
| on day after the | | | | |
| treatment | | | | |
| Toltrazuril in mg/l | 1 | 2180 | 650 | 2281 |
| on day after the | | | | |
| treatment | | | | |
| Toltrazuril in mg/l | 7 | 9246 | 2042 | 2275 |
| on day after the | | | | |
| treatment | | | | |
| Toltrazuril in mg/l | 13 | 2194 | 1806 | 702 |
| on day after the | | | | |
| treatment | | | | |
| Toltrazuril in mg/l | 21 | 462 | 371 | 430 |
| on day after the | | | | |
| treatment | | | | |
| Ponazuril in mg/l on | 0 | <LoQ | <LoQ | <LoQ |
| day after the | | | | |
| treatment | | | | |
| Ponazuril in mg/l on | 1 | 156 | 44 | 146 |
| day after the | | | | |
| treatment | | | | |
| Ponazuril in mg/l on | 7 | 9715 | 2735 | 2615 |
| day after the | | | | |
| treatment | | | | |
| Ponazuril in mg/l on | 13 | 4506 | 3440 | 1905 |
| day after the | | | | |
| treatment | | | | |
| Ponazuril in mg/l on | 21 | 1394 | 733 | 1172 |
| day after the | | | | |
| treatment | | | | |

<LoQ = below the limit of quantitation [25 μg/l])

TABLE 2
Serum values of toltrazuril/ponazuril in rabbits

| | | Animal 1 | Animal 2 | Animal 3 |
|---|---|---|---|---|
| Toltrazuril in mg/l on | 1 | 9.06 | 8.67 | 9.67 |
| day after the treatment | | | | |
| Toltrazuril in mg/l on | 2 | 10.39 | 6.03 | 8.03 |
| day after the treatment | | | | |
| Toltrazuril in mg/l on | 3 | 6.66 | 5.07 | 6.70 |
| day after the treatment | | | | |
| Ponazuril in mg/l on | 1 | 1.79 | 1.38 | 1.38 |
| day after the treatment | | | | |
| Ponazuril in mg/l on | 2 | 4.72 | 2.63 | 2.83 |
| day after the treatment | | | | |
| Ponazuril in mg/l on | 3 | 5.03 | 4.07 | 3.42 |
| day after the treatment | | | | |

The present studies demonstrate that toltrazuril, after pour-on application, is percutaneously absorbed, and metabolised, both in rabbits and in calves.

B. Activity of Toltrazuril Pour-On Against Artificially Provoked Infections in Suckling Pigs with Isospora suis:

3 groups with 4 to 11 animals were formed. Group A was infected with oocysts and treated with the toltrazuril pour-on formulation of Example 10. Group B was not infected, but treated in the same manner, the dosage rate of group A and B being in each case 20 mg/kg body weight. Group C was infected with oocysts, but not treated with toltrazuril. The success of the treatment was determined by monitoring the live weight of the animals. To this end, the animals were weighed on different days, and the weight gain of the individual animals was determined The results are shown in Table 3.

Table 3: Average live weight per piglet (g)

TABLE 3
Average live weight per piglet (g)

| Group | Day 0 | Day 7 | Day 14 | Day 21 | Day 28 |
|---|---|---|---|---|---|
| Infected, treated | 1702 | 3000 | 3635 | 7097 | 9337 |
| Group A (n* = 4) | | | | | |
| Not infected, but treated | 1829 | 3426 | 5327 | 7363 | 9903 |
| Group B (n* = 7) | | | | | |
| Infected, but untreated | 1773 | 3015 | 3708 | 5725 | 7815 |
| Group C (n* = 11) | | | | | |

*at the beginning of the study

As can be seen from Table 3, the weight of the infected animals of Group A, which have been treated with toltrazuril pour-on, is on average 1520 g higher than that of the infected, but untreated animals of Group C. These data show clearly that the formulation is effective in the desired form.

1. A method of transdermallv treating an animal or human with a coccidian infection comprising administering to said animal or human a composition comprinq lice of triazines of the formula (I) or (II) 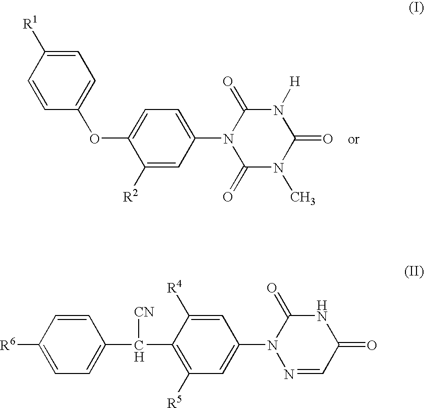
where
R¹ represents R³—SO₂— or R³—S—,
R² represents alkyl, alkoxy, halogen or SO₂N(CH₃)₂ and
R³ represents haloalkyl
R⁴ and R⁵ independently of one another represent hydrogen or Cl and
R⁶ represents fluorine or chlorine and their physiologically acceptable salts.

2. The method of claim 1, wherein the animal is selected from the group consisting of pigs, sheep, cattle, dogs, cats, turkeys, geese, and pigeons.

3. The method of claim 1, wherein the animal is a pigs.

4. (canceled)

5. The method of claim 1, wherein the animal or human is administered a dose which is approximately the same or lower than in the case of oral administration.

6. The method of claim 1, wherein the triazine is toltrazuril.

7. The method of claim 1, wherein the triazine is ponazuril.
